SEQUENCE LISTING (9 sequences)

SEQ ID NO: 1 - DNA, 20 bp, Artificial
  Primer
tcccggctacaacaacacgt

SEQ ID NO: 2 - DNA, 21 bp, Artificial
  Primer
agatgtcattgtagcggaccg

SEQ ID NO: 3 - DNA, 24 bp, Artificial
  Probe
ccagatcaatgcccgcacagagct

SEQ ID NO: 4 - DNA, 21 bp, Artificial
  Primer
tggcaaggttaagcctttcaa

SEQ ID NO: 5 - DNA, 20 bp, Artificial
  Primer
atctgcgtgttctggatccc

SEQ ID NO: 6 - DNA, 34 bp, Artificial
  Probe
atgacgaacagtttctggaagcttttgtcatctt

SEQ ID NO: 7 - DNA, 20 bp, Artificial
  Primer
tccaccttccagcagatgtg

SEQ ID NO: 8 - DNA, 21 bp, Artificial
  Primer
ctagaagcatttgcggtggac

SEQ ID NO: 9 - DNA, 29 bp, Artificial
  Probe
atcagcaagcaggcagtatgacgagtccg

(19) United States
(12) Patent Application Publication
(10) Pub. No.: US 2004/0266736 A1
(43) Pub. Date: Dec. 30, 2004

(54) Regulation of cgmp-specific phosphodiesterase 9a

(76) Inventors: Frank Wunder, Wuppertal (DE); Peter Ellinghaus, Wuppertal (DE)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/495,638

(22) PCT Filed: Nov. 11, 2002
(86) PCT No.: PCT/EP02/12550

(30) Foreign Application Priority Data
Nov. 15, 2001 (DE) 101 56 249.7

(51) Int. Cl. A61K 31/66; A61K 31/519

(52) U.S. Cl. 514/114; 514/252.16

(57) ABSTRACT

The invention relates to the use of PDE9A inhibitors for producing a medicament for the treatment and/or prophylaxis of coronary heart disease, especially stable and unstable angina pectoris, acute myocardial infarction, myocardial infarction prophylaxis, sudden heart death, heart failure, high blood pressure and the sequelae of atherosclerosis.

The invention relates to the use of PDE9A inhibitors for producing a medicament for the treatment and/or prophylaxis of coronary heart disease, especially stable and unstable angina pectoris, acute myocardial infarction, myocardial infarction prophylaxis, sudden heart death, heart failure, high blood pressure and the sequelae of atherosclerosis.

As a ceaselessly working hollow muscle, the heart requires a particularly intensive supply of oxygen to cover its energy requirements. Interferences with supply therefore relate primarily to oxygen transport, which may be inadequate if the adaptability of the blood flow is reduced. An increase in oxygen consumption can be covered only by an increase in the blood flow to the heart.

In coronary heart diseases such as stable and unstable angina pectoris, heart failure, myocardial infarction, sudden heart death, and the sequelae of atherosclerosis, an adequate blood flow to parts of the cardiac tissue is no longer ensured, and tissue ischaemias occur, leading to necrosis and apoptosis in the affected areas. This results in myocardial dysfunction which may develop as far as heart failure.

Therapeutic methods and active ingredients which improve coronary blood flow and thus the oxygen supply, but also those which reduce the oxygen consumption, are suitable for treating symptoms of the abovementioned disorders.

These include dilatation of larger coronary vessels, reduction in the extravascular component of the coronary resistance, reduction of the intramyocardial wall tension, and dilatation of the arteriolar resistance vessels in the systemic circulation.

Substances and methods leading to an increase in the coronary flow in the heart and/or to a reduction in blood pressure can be utilized therapeutically (Forth, Henschler, Rummel; Allgemeine und spezielle Pharmakologie und Toxikologie; Urban & Fischer Verlag (2001), Munich)

The effects described above can be controlled via the intracellular concentration of the so-called second messengers cyclic adenosine monophosphate (cAMP) and cyclic guanosine monophosphate (cGMP). The intracellular concentration of cGMP is increased by stimulation of the soluble and membrane-bound guanylate cyclases. The intracellular concentration of cAMP can be modulated by activating so-called G protein-coupled receptors. Activation of these receptors leads to activation of G proteins and thus to activation or inhibition of adenylate cyclase.

So-called phosphodiesterases are involved in the degradation of intracellular cAMP and cGMP. The phosphodiesterases are divided into eleven different classes according to their biochemical and pharmacological properties (Soderling and Beavo, Current Opinion in Cell Biology, (2000) 174-179; Francis et al., Prog. Nucleic Acid Res. Mol. Biol. (2000) 1-52).

Phosphodiesterase 9A (PDE9A) is a cGMP-specific phosphodiesterase. The enzyme has a Km (Michaelis-Menten constant) of 70 nM (Soderling et al., J. Biol. Chem. (1998) 15553-15558), which is the lowest known Km for cGMP of all known phosphodiesterases. PDE9A is therefore involved in the maintenance and regulation of the basal intracellular cGMP levels.

The DNA and protein sequences for phosphodiesterase 9A are known for the mouse (Soderling et al., J. Biol. Chem. (1998) 15553-15558) and humans (Fisher et al., J. Biol. Chem. (1998) 15559-15564; Guipponi et al., Hum. Gen. (1998) 386-392). To date, four splice variants of PDE9A have been identified (Guipponi et al. Hum. Gen. (1998) 386-392).

PDE9A expression was detectable in mice in particular in the kidney, but also, more weakly, in the lung and liver (Soderling et al., J. Biol. Chem. (1998) 15553-15558). In humans, strong expression was shown in particular in the spleen, kidney, intestine, prostate and brain, but weaker expression was also detected in other organs such as lung, liver, heart and pancreas (Fisher et al., J. Biol. Chem. (1998) 15559-15564; Guipponi et al., Hum. Gen. (1998) 386-392).

It has surprisingly now been found in the quantitative analysis of PDE9A mRNA expression in humans that there is pronounced expression of PDE9A in human coronary arteries (FIGS. 1 and 2).

PDE9A expression in the human coronary artery is moreover surprisingly in fact about 2.7 times higher than the expression of phosphodiesterase 5A in this tissue (FIG. 2).

Phosphodiesterase 5A is known from the literature to be involved in the blood supply to the heart. It has been shown that administration of PDE5A inhibitors leads to relaxation of coronary vessels (Traverse et al., Circulation (2000) 2997-3002).

The high, also in comparison with PDE5A, expression of PDE9A in the human coronary artery, and the extremely high affinity of PDE9A for cGMP (Km 70 nM) now indicate that phosphodiesterase 9A has a very significant role in the contraction and relaxation of coronary arteries and thus in controlling the blood supply to the heart.

PDE9A expression in blood vessels thus also indicates a role of PDE9A in controlling the blood pressure and regulating the peripheral blood flow.

The effect of PDE9A inhibitors on the coronary flow can be investigated on the isolated perfused Langendorff heart. A PDE9A inhibitor reduces the perfusion pressure in the Langendorff rat heart.

Since expression of human phosphodiesterase 9A in coronary arteries is a condition for the use of active ingredients which inhibit PDE9A in patients with coronary heart disease, this result creates the basis for a novel therapeutic approach.

On the basis of this novel result, we came to the conclusion that substances which inhibit phosphodiesterase 9A can, because of the increase, resulting therefrom, in the intracellular cGMP concentration and the dilatation, associated therewith, of blood vessels, specifically coronary arteries (and the increase, associated therewith, in the coronary flow), be employed for the treatment and/or prophylaxis of stable and unstable angina pectoris, acute myocardial infarction, myocardial infarction prophylaxis, heart failure, sudden heart death, and high blood pressure, peripheral blood flow impairments and the sequelae of atherosclerosis in humans.

The present invention therefore relates to the use of phosphodiesterase 9A inhibitors for producing a medicament for the treatment and/or prophylaxis of the abovementioned diseases.

Inhibitors for the purpose of the invention are all substances which prevent (inhibit) activation or the biological activity of the enzyme. The inhibition can be measured for example in the cGMP assay described below. Particularly preferred inhibitors are low molecular weight substances.

Inhibition means for phosphodiesterase 9A a decrease of at least 10% in the activity or an increase of at least 10% in the intracellular cGMP concentration in a cell containing the phosphodiesterase 9A. Inhibitors can be tested on PDE9A purified from suitable tissue or recombinantly expressed and purified. It is additionally possible to determine the intracellular cGMP concentration in a cell containing the phosphodiesterase 9A. These cells are preferably cells from the smooth muscles of vessels or from cell lines which recombinantly express PDE9A.

Moreover preferred PDE9A inhibitors are those which inhibit in the activity assay indicated below with an IC₅₀ of 1 μM, preferably less than 0.1 μM.

The PDE9A inhibitors of the invention are preferably unable to cross the blood/brain barrier, and act systemically and not centrally.

### BRIEF DESCRIPTION OF THE FIGURES

FIG. 1.) Relative expression of human phosphodiesterase 9A in human tissues (see Table 1 for data)

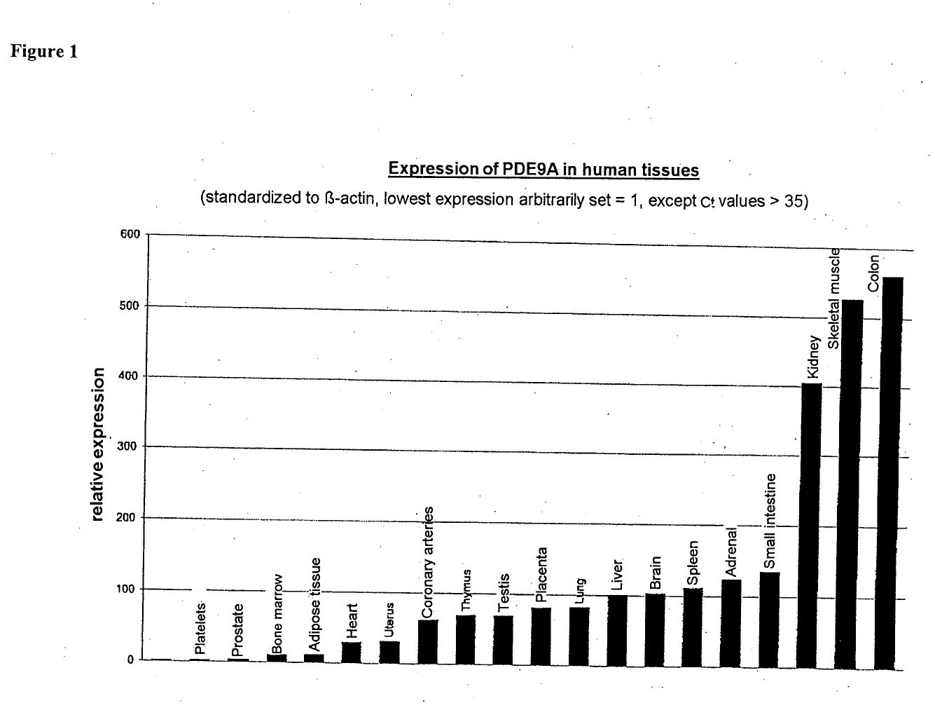

FIG. 2.) Comparison of the relative expression of human PDE9A with PDE5A in the human coronary artery

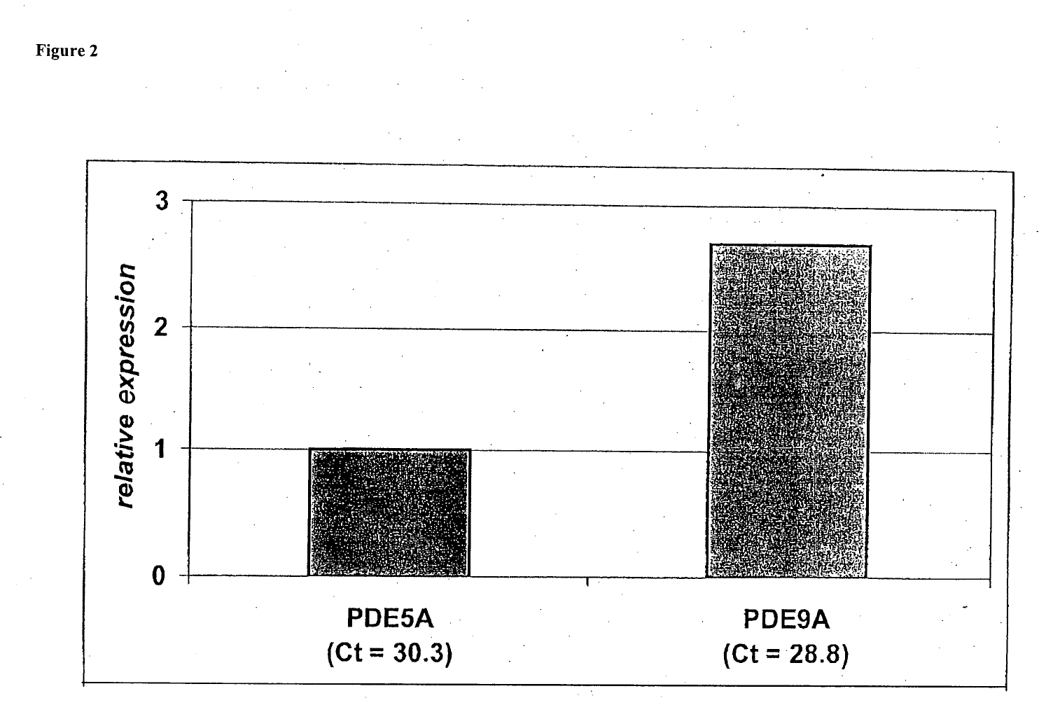

### INHIBITION OF cGMP-SPECIFIC PDE9A

The effect of PDE9A inhibitors is tested on the isolated enzyme. It is possible to use for this purpose for example the phosphodiesterase [³H]cGMP SPA enzyme assay kit from Amersham. The test is carried out in accordance with the manufacturer's instructions.

To characterize test substances, a suitable dilution of the enzyme, various concentrations of the inhibitor (serial dilutions typically of 10⁻⁹−10⁻⁵ M), and [³H]cGMP (0.05 μCi per mixture) are incubated in a 96-well microtiter plate at 30° C. for 15 min. After the reaction has been stopped, the “SPA beads” are added and the microtiter plate is shaken for 30 seconds. After 60 min, the measurement takes place with the aid of a scintillation counter suitable for microtiter plates (e.g. 1450 MicroBeta, Wallac).

The IC₅₀ of the effect of a PDE9A inhibitor is the value at which 50% of the cGMP degradation by the PDE9A is inhibited.

Quantification of PDE9A and PDE5A mRNA Expression in Human Tissues

The relative expression of PDE9A in human tissues is measured by quantifying the mRNA values of the real-time polymerase chain reaction (PCR) (so-called TaqMan PCR, Heid et al., Genome Res., 1996, 6 (10), 986-994). Compared with conventional PCR, the real-time PCR has the advantage of more accurate quantification through the introduction of an additional fluorescence-labelled oligonucleotide. This so-called probe contains at the 5′ end the fluorescent dye FAM (6-carboxyfluorescein) and at the 3′ end the fluorescence quencher TAMRA (6-carboxytetramethylrhodamine). During the polymerase chain reaction, the fluorescent dye FAM is cleaved off the probe by the 5′-exonuclease activity of the Taq polymerase in the TaqMan PCR, and thus the previously quenched fluorescence signal is obtained.

The template used for the PCR is commercially obtained total RNA (from Clontech). In the case of the coronary arteries, small pieces (approx. 0.5 g) of explanted heart are obtained from the German Cardiac Centre in Berlin and, after homogenization, the total RNA is isolated therefrom by phenol/chloroform extraction. 1 μg portions of total RNA are incubated with 1 unit of DNase I (from Gibco) at room temperature for 15 min to remove genomic DNA contamination. The DNase I is inactivated by adding 1 μl of EDTA (25 mM) and then heating at 65° C. (10 min). Subsequently, the cDNA synthesis is carried out in accordance with the instructions for the “SUPERSCRIPT-II RT cDNA synthesis kit” (from Gibco) in the same reaction mixture, and the reaction volume is made up to 200 μl with distilled water.

For the PCR, 7.5 μl of primer/probe mix and 12.5 μl of TaqMan Universal Master Mix (from Applied Biosystems) are added to each 5 μl portion of the diluted cDNA solution. The final concentration of the primers is 300 nM in each case, and that of the probe is 150 nM. The sequence of the forward and reverse primers for PDE9A is: 5′-TCCCGGCTACAACAACACGT-3′ and 5′-AGATGTCATTGTAGCGG-ACCG-3′, the sequence of the fluorescence-labelled probe 5′-6FAM-CCAGATCAATGCCCGCACAGAGCT-TAMRA-3′. The location of the amplicon is chosen so that all four described splice variants of the PDE9A mRNA (PDE9A₁₋₄) are detected. For PDE5A, the sequence of the forward primer is: 5′-TGGCAAGGTTAAGCCTTTCAA-3′, that of the reverse primer is: 5′-ATCTGCGTGTTCTGGATCCC-3′ and the sequence of the probe is 5′-FAM-ATGACGAACAGTTTCTGGAAGCTTTTGTCATCTT-TAMRA-3′. Once again, the location of the amplicon on the mRNA is chosen so that both splice variants (PDE5A₁₋₂) are detected.

The PCR takes place on an ABI prism SDS-7700 apparatus (from Applied Biosystems) in accordance with the manufacturer's instructions. 40 cycles are carried out as standard for this purpose. A so-called threshold cycle (Ct) is obtained for each tissue and for each probe. The Ct corresponds to the cycle in which the fluorescence intensity of the liberated probe reaches 10 times the background signal. Thus, a lower Ct means an earlier start of amplification, i.e. more mRNA present in the original sample. To compensate for any variations in the cDNA synthesis, the expression of a so-called housekeeping gene is also analyzed in all the tissues investigated. The strength of expression of this gene ought to be approximately the same in all tissues. For this purpose, β-actin is used to standardize the PDE9A and PDE5A expression. The sequence of the forward and reverse primers for human cytosolic β-actin is: 5′-TCCACCTTCCAGCAGATGTG-3′, and 5′-CTAGAAGCATTTGCGGTGGAC-3′ respectively, and the sequence of the probe 5′-6FAM-ATCAGCAAGCAGGCAGTATGACGAGTCCG-TAMRA-3′. The data are analyzed by the so-called ddCt method in accordance with the instructions for the ABI prism SDS 7700 (from Applied Biosystems). For graphical representation of the tissue distribution of the PDE9A mRNA, the level of expression of the tissue with the highest Ct(=lowest expression) is arbitrarily set equal to 1 and all the other tissues are standardized thereto.

Langendorff Rat Heart

The heart is rapidly removed after opening the chest cavity of anaesthetized rats and is introduced into a conventional Langendorff apparatus. The coronary arteries are subjected to constant-volume (10 ml/min) perfusion, and the perfusion pressure arising thereby is recorded via an appropriate pressure transducer. A decrease in the perfusion pressure in this arrangement corresponds to a relaxation of the coronary arteries. At the same time, the pressure (LVP) developed by the heart during each contraction is measured via a balloon introduced into the left ventricle, and a further pressure transducer. The rate at which the isolated heart beats is found by calculation from the number of contractions per unit time. The test substances are added in a series of increasing concentrations (normally 10⁻⁹ M to 10⁻⁶ M) with the aid of a perfusor.

PDE9A Inhibitor Formulations

The PDE9A inhibitors can be converted in a known manner into the usual formulations such as tablets, coated tablets, pills, granules, aerosols, syrups, emulsions, suspensions and solutions, using inert, nontoxic, pharmaceutically suitable carriers or solvents. The therapeutically active compound should be present in each of these in a concentration of from 0.5 to 90% by weight of the complete mixture, e.g. in amounts sufficient to achieve the indicated dosage range.

The formulations are produced for example by extending the active ingredients with solvents and/or carriers, where appropriate with use of emulsifiers and/or dispersants, it being possible for example if water is used as diluent where appropriate to use organic solvents as auxiliary solvents.

Administration takes place in a conventional way, preferably orally, transdermally, intravenously or parenterally, in particular orally or intravenously. It can, however, also take place by inhalation through the mouth or nose, for example with the aid of a spray, or topically through the skin.

It has generally proved advantageous to administer amounts of about 0.001 to 10 mg/kg, on oral administration preferably about 0.005 to 3 mg/kg, of body weight to achieve effective results.

It may nevertheless be necessary where appropriate to deviate from the amounts mentioned, specifically as a function of the body weight or the nature of the administration route, the individual response to the medicament, the nature of its formulation and the time or interval over which administration takes place. Thus, it may in some cases be sufficient to make do with less than the aforementioned minimum amount, whereas in other cases the upper limit mentioned must be exceeded. Where larger amounts are administered it may be advisable to distribute these into a plurality of single doses over the day.

TABLE 1

| PDE9A | RE | Ct | Ct βActin |
|---|---|---|---|
| Macrophage | 0.12 | 36.51 | 17.66 |
| Platelet | 1.00 | 34.96 | 19.18 |
| Prostate | 2.20 | 31.18 | 16.54 |
| Bone marrow | 9.06 | 31.63 | 19.03 |
| Adipose tissue | 10.78 | 32.47 | 20.12 |
| Heart | 29.04 | 30.03 | 19.11 |
| Uterus | 30.70 | 28.19 | 17.35 |
| Coronary art. | 61.39 | 30.7 | 20.46 |
| Thymus | 68.59 | 28.22 | 18.38 |
| Testis | 68.59 | 27.82 | 18.14 |
| Placenta | 80.45 | 28.03 | 18.58 |
| Lung | 81.57 | 27.5 | 18.07 |
| Liver | 100.43 | 30.19 | 21.06 |
| Brain | 102.54 | 28.58 | 19.48 |
| Spleen | 111.43 | 26.85 | 17.87 |
| Adrenal | 123.64 | 28.38 | 19.55 |
| Small intestine | 134.36 | 27.45 | 18.74 |
| Kidney | 404.50 | 26.61 | 19.49 |
| Skeletal m. | 526.39 | 25.07 | 18.33 |
| Colon | 560.28 | 24.95 | 18.3 |

1. A method of treating coronary heart disease, comprising administering to a patient in need thereof an effective amount of a PDE9A inhibitor.

2. A method of treating high blood pressure, comprising administering to a patient in need thereof an effective amount of a PDE9A inhibitor.

3. A method of treating peripheral occlusive diseases, comprising administering to a patient in need thereof an effective amount of a PDE9A inhibitor.

4. A method of treating atherosclerosis, comprising administering to a patient in need thereof an effective amount of a PDE9A inhibitor.

5. The method of claim 1, wherein the coronary heart disease is stable or unstable angina pectoris, acute myocardial infarction, myocardial infarction prophylaxis, sudden heart death or heart failure.

6. The method of claim 1, wherein the PDE9A inhibitor has an IC₅₀ of less than 1 μM.

7. The method of claim 1, wherein the PDE9A inhibitor has an IC₅₀ of less than 100 nM.
